(19) United States
(12) Patent Application Publication
(10) Pub. No.: US 2009/0209987 A1
(43) Pub. Date: Aug. 20, 2009

(54) SNARE WITH CAPTURE-AREA ENHANCEMENT

(76) Inventors: Eric D. Mathews, Needham, MA (US); Paul J. Dobson, Hanover, MA (US); Jeffry Coats, Plymouth, MA (US)

Correspondence Address: FISH & RICHARDSON PC, P.O. BOX 1022, MINNEAPOLIS, MN, 55440-1022, US

(21) Appl. No.: 11/815,273

(22) PCT Filed: Feb. 1, 2006
(86) PCT No.: PCT/US06/03661

§ 371 Date: Mar. 26, 2008

Related U.S. Application Data
(60) Provisional application No. 60/648,891, filed on Feb. 1, 2005.

(51) Int. Cl. A61B 17/22

(52) U.S. Cl. 606/159

(57) ABSTRACT

A surgical snare for ensnaring an object in a patient includes a flexible section that selectively switches between an insertion shape and an ensnaring shape; and a capture-area enhancer deployed when the flexible section assumes the ensnaring shape. An actuator coupled to the flexible section causes the flexible section to transition between the insertion shape and the ensnaring shape.

### RELATED APPLICATIONS

This application claims priority under 35 U.S.C. § 119 to U.S. Provisional Patent Application Ser. No. 60/648,891, filed Feb. 1, 2005, the entire contents of which are hereby incorporated by reference.

### TECHNICAL FIELD

This invention relates to surgical instruments, and more particularly to endovascular snaring instruments.

### BACKGROUND OF THE INVENTION

A clot in a patient's blood vessel poses grave risks for those portions of a patient's anatomy that are downstream from the clot. Because a clot can inhibit blood flow, cells that rely for their nourishment on blood passing through the obstructed vessel can die. If those cells are particularly essential to life, such as cells associated with the brain or the heart, the patient can also die.

When a blood clot is small relative to the blood vessel, or where the clot is obstructing a relatively minor blood vessel, the patient is generally in no immediate danger. Nevertheless, there does exist the more insidious danger of the blood clot becoming detached and coming to rest again in another blood vessel in which its obstructive effect is less benign. Additionally, there exists the danger that small blood clots migrating through the circulatory system will coalesce with a stationary clot and thereby cause it to enlarge by accretion. When this occurs, a clot of negligible size can grow into a significant obstruction. This growth can occur rapidly because as the clot grows, it introduces more turbulence into the blood flow. This turbulence tends to break up thrombocytes as they pass through the turbulent zone, thereby causing additional clotting.

Conventional methods of removing a blood clot rely on the introduction of medicaments, such as enzymes, that dissolve blood clots. Because the enzymes, such as streptokinase, are introduced into the bloodstream, their effects are systemic rather than local. In addition, the process of dissolving a clot is a time-consuming one during which the patient continues to be in some danger.

Mechanical methods of removing a blood clot have been generally unsuccessful because of the fragility of the clot. When disturbed by a conventional mechanical device, a clot can easily fragment into smaller clots, each of which then begins migrating through the blood stream before settling at an unpredictable location.

### SUMMARY

The invention is based on the recognition that the effective capture area of a snare can be increased by incorporating certain structures on that portion of the snare that is intended to ensnare an object.

In one aspect, the invention features a snare including a support defining an axis, a capture-area enhancer coupled to a flexible section on the support; a core-wire extending along the axis and anchored to the flexible section, and an actuator engaged to a proximal end of the core-wire. The core-wire has a first state, in which it defines a first path and a second state, in which it defines a second path. The actuator selectively causes the core-wire to transition between the first state and the second state.

In some embodiments, the first state of the core-wire is a relaxed state and the second state of the core-wire is a tensioned state.

Embodiments also include those in which the capture-area enhancer includes a plurality of threads attached to the flexible section, those in which the capture-area enhancer includes a sock, for example a nitinol mesh sock or a polymer sock, that covers the flexible section, those in which the capture-area enhancer includes a suture wound on the flexible section, those in which the capture-area enhancer includes a spiny coating on the flexible section, and those in which the capture-area enhancer includes a hydrogel coating on the flexible section.

Additional embodiments include those having a drug-releasing polymer coating on the flexible section.

Yet other embodiments include those having an additional core-wire extending along the axis and anchored to the flexible section and an additional actuator engaged to a proximal end of the additional core-wire. The additional core-wire has a first state, in which it defines an additional first path, and a second state, in which it defines an additional second path. The additional actuator selectively causes the additional core-wire to transition from the first state to the second state.

Other embodiments include a power source in electrical communication with the core wire. The power source provides current to heat the core wire, thereby causing the core wire to transition between the first and second state.

In another aspect, the invention features a snare including a support defining an axis, and a capture area enhancer coupled to a flexible distal section of the support. The flexible distal section has an uncompressed state and a compressed state, and defines a first path relative to the axis when in the compressed state. The snare also includes a core-wire extending along the axis and anchored to the flexible distal section. The core-wire has a relaxed state and a tensioned state, and defines a second path relative to the axis when in the relaxed state. The snare also includes an actuator engaged to a proximal end of the core-wire for selectively applying a tensile force thereto. The tensile force causes the core-wire to transition from its relaxed state, in which the flexible distal section is in its uncompressed state, to its tensioned state, in which the flexible distal section is in its compressed state.

Embodiments of the invention include those in which the capture-area enhancer includes a plurality of threads attached to the distal section, those in which the capture-area enhancer includes a sock covering the distal section, those in which the capture-area enhancer includes a hydrogel coating on the distal section, those in which the capture-area enhancer includes a suture wound on the distal section, and those in which the capture-area enhancer includes a spiny coating on the distal section.

In yet another aspect, the invention features a surgical snare for ensnaring an object in a patient. The snare includes a flexible section that selectively switches between an insertion shape and an ensnaring shape; and a capture-area enhancer configured to deploy when the flexible section assumes the ensnaring shape.

Other features and advantages of the invention will be apparent from the following detailed description, and from the claims. The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is an illustration of a snare according to the invention in its extended state.

FIG. 2 is an illustration of the snare in FIG. 1 in its coiled state.

FIG. 3 is a cut-away view of the snare of FIG. 1.

FIG. 4 is a cross-section of the coil section of the snare in FIG. 1.

FIG. 5 is a cross-section of the coil section in FIG. 4 with the core-wire in its extended state.

FIGS. 6A-F show capture-area enhancers at the distal section of the snare.

FIGS. 7 shows a snare with multiple core wires.

FIGS. 8A-8D shown four paths defined by the snare in FIG. 7.

FIGS. 9A-9B show multiple core-wire snares in which the core-wires have different properties.

FIGS. 10A-10C show multiple core-wire snares in which the core wires have different lengths.

FIGS. 11A-11B show coil section windings at the distal end of the snare.

FIGS. 12A-12B show states of a guide catheter having an expandable distal segment.

FIGS. 13-14 show snares with variable-strength distal segments.

### DETAILED DESCRIPTION

Surgical instruments described herein use an inhomogeneous core-wire that, when subjected to a pulling force, stretches by different amounts at different locations. At least one portion of the wire has a relaxed state in which it takes the shape of a coil and a tensioned state in which it becomes straight. This portion of the wire is attached to and controls the shape of a flexible portion of the instrument. The tension on the core-wire is controlled by a surgeon selectively pulling and releasing the wire.

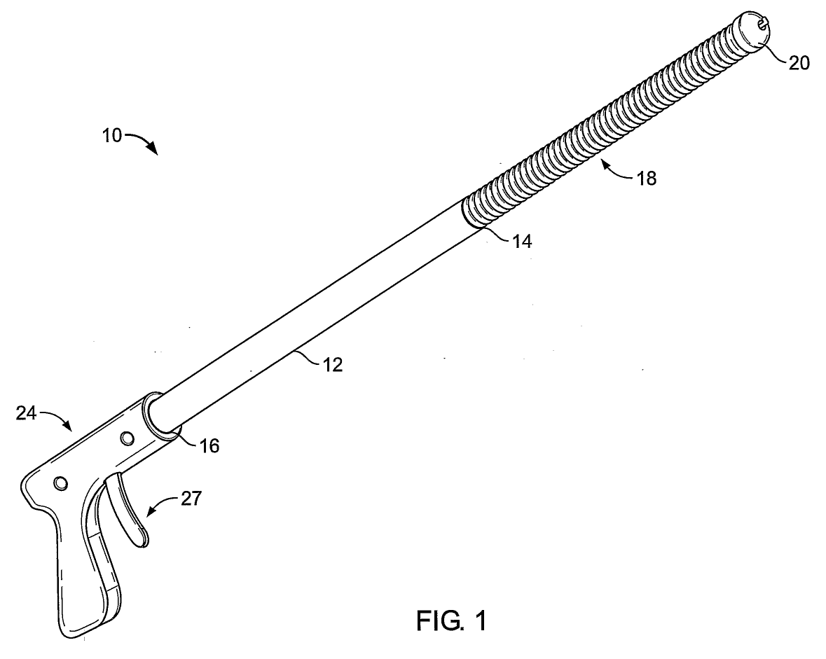

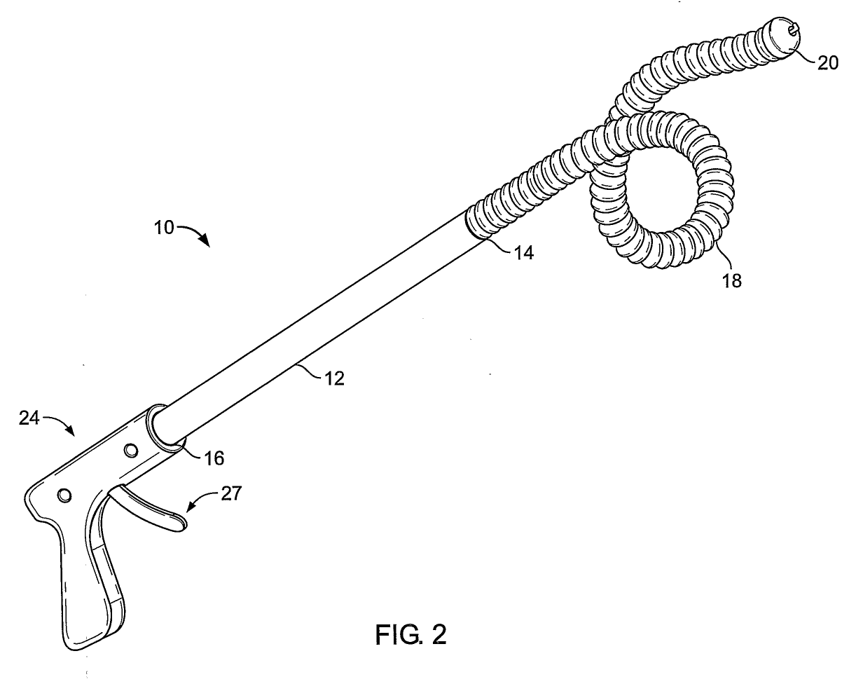

Referring to FIG. 1, a surgical instrument 10 incorporating the principles of the invention includes a cannula 12 extending along an axis between a distal end 14 and a proximal end 16. A flexible section 18 is mounted at the distal end of the cannula 12. The flexible section 18 is capped at its distal end by an end-cap 20. Attached to the proximal end 16 of the cannula 12 is an actuator 24 operable by a surgeon to switch the flexible section 18 between an extended state, shown in FIG. 1, and a coiled state, shown in FIG. 2.

The cannula 12 in the illustrated embodiment is tubular. However, this is not a requirement. The function of the cannula 12 is to support the flexible section 18 when the surgeon applies a force sufficient to toggle the flexible section 18 into its extended state. The cannula 12 can be a metal, such as nitinol, stainless steels, or MP35N®. The cannula 12 can also be made from a polymer, constructed from polyimide, any of the various nylons and polytetrafluoroethylenes such as those sold under the trade name TEFLON®; or it can be a composite tube made from any number of polymers. In addition, the cannula 12 can encapsulate a metallic spring, braid, or similar structure. In some embodiments, the cannula 12 is integral with the flexible section 18.

In the particular embodiment shown by FIGS. 1 and 2, the flexible section 18 is a segmented structure capable of articulation between segments. However, the flexible section 18 can also be any flexible section capable of freely making the required transition between two or more distinct states, such as the coiled state of FIG. 2 and the extended state of FIG. 1. A preferred flexible section 18 has an equilibrium compressed state in which it defines a path corresponding to that shown in FIG. 1. In the illustrated embodiment, the flexible section 18 and the cannula 12 are tubular structures that can be coated with a hydrophilic and biocompatible composite material such as a hydrogel. A suitable outer diameter for general intra-vascular use is approximately 0.014 inches.

To enable a surgeon to track the position of the instrument 10 within the body, the flexible section 18 can be made of a radio-opaque material such as Pt, W, Ir, Tn, Au, Ag, or an alloy thereof. Alternatively, the flexible section 18 can be made of coilable polymer, stainless steel, MP35N®, or similar a substance, in which case the flexible section 18 is coated with a radio-opaque coating. The flexible section 18 may be a close wound coil, with or without preload, or it may be an open wound coil. The flexible section 18 can also include baffles, bellows, or any such flexible and compressible member.

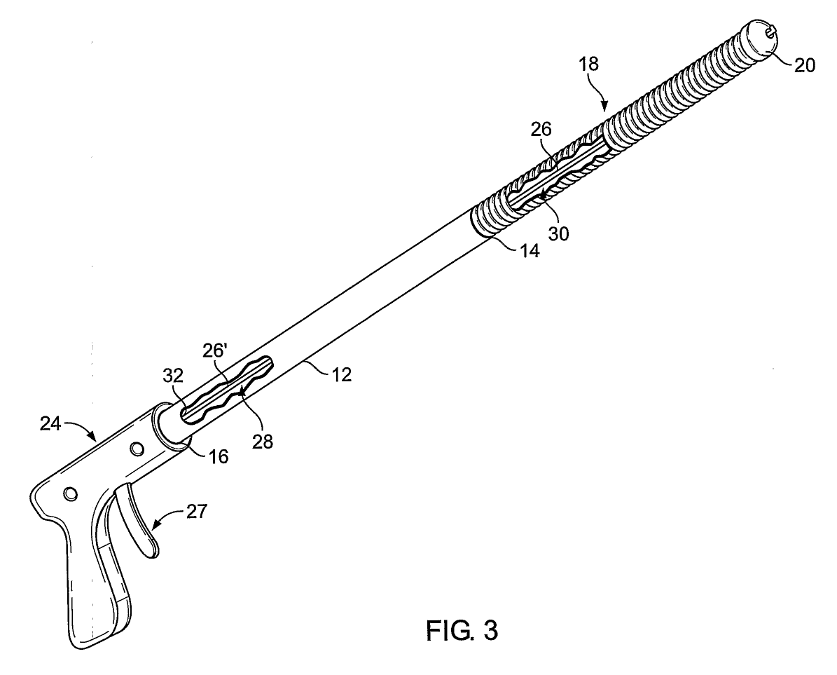

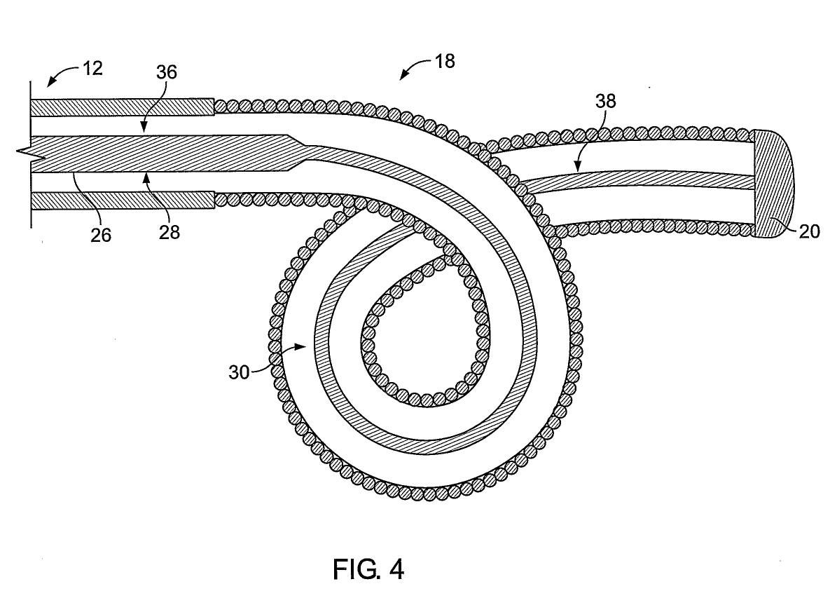

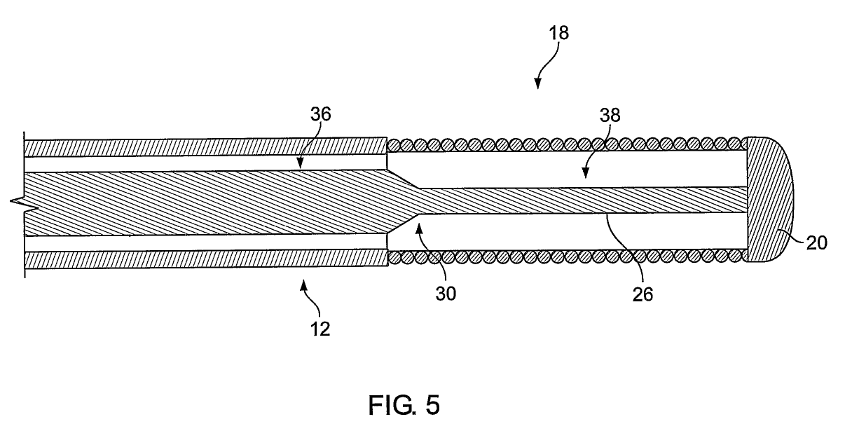

A cut-away view of the surgical instrument 10 shown in FIG. 3, reveals a portion of the structure that enables the surgeon to toggle the flexible section 18 between its coiled state and its extended state. As shown in FIG. 3, a core-wire 26 extending from the actuator 24 to the end-cap 20 passes through a cannula lumen 28 and a coil-section lumen 30. The core-wire 26 has a proximal end 32 operably connected to the actuator 24 and a distal end 36 anchored to the flexible section 18. In one embodiment, an end-cap 20 functions as an anchoring element on the flexible section 18 and the distal end 36 is anchored to the end-cap 20, as shown in FIGS. 4 and 5.

The core-wire 26 is preferably made of a shaped-memory and super-elastic alloy. Such a metal has the property that when deformed and heated past a critical temperature, it “remembers” its deformed shape. When cooled and subjected to further deformation, such a wire springs back to this remembered shape. A suitable super-elastic metal from which the core-wire can be manufactured is a nickel-titanium alloy sold under the trade name nitinol. In the case of nickel-titanium alloy, the critical temperature is in the neighborhood of 700 degrees Fahrenheit. However, other materials can be used, each with a different critical temperature.

Because the core-wire 26 is anchored to both the end-cap 20 of the flexible section 18 and to the actuator 24, and because the flexible section 18 is flexible, the core-wire 26 and the flexible section 18 follow the same path relative to the axis. When the core-wire 26 is in its coiled state, as shown in FIGS. 2 and 4, the flexible section 18 is in an uncompressed state, in which it follows the coiled path defined by the core-wire 26. When the core-wire 26 is in its extended state, as shown in FIGS. 1 and 5, the flexible section 18 reverts to a compressed state, in which it extends along the axis.

As shown in FIGS. 4 and 5, the core-wire 26 has two sections: a proximal section 36 that extends through the cannula lumen 28 and attaches to the actuator 24; and a super-elastic distal section 38 that extends through the coil lumen 30 and attaches to the end-cap 20. The proximal section 36 has a yield force that exceeds that of the distal section 38. This enables the distal section 38 to experience more strain for a given tensile force on the core-wire 26 than the proximal section 36.

For a given tensile force, the extent to which a material is stretched depends on its cross-sectional area. This phenomenon is familiar to anyone who has pulled on a piece of taffy and observed that the thin section is far easier to stretch than the thick section. The extent to which the material is stretched is referred to as “strain.” The cause of strain is “stress,” a quantity which, like pressure, is a force per unit area. Stress can be thought of as pressure acting in the opposite direction. Whereas an applied pressure tends to compress a material, an applied stress tends to stretch a material.

For many materials, no significant strain occurs until a threshold of tensile force is reached. Once that threshold is reached, the material responds readily to additional force. This threshold at which a material begins to respond to an applied tensile force is referred to as the “yield force” of the material.

As noted above, the core-wire 26 transitions from a coiled state to an extended state because the distal section 38 of the core-wire 26 and the proximal section 36 of the core-wire 26 have different yield forces. This difference in yield forces can be achieved by having a core-wire 26 in which the distal section 38 has a smaller cross-sectional area than the proximal section 36. A differential yield force in the core-wire 26 can also be achieved by having the distal and proximal sections 38, 36 of the core-wire 26 be made of different materials. In such an embodiment, the proximal section 36 would be made of a first material that experiences a negligible amount of strain for a given applied stress. The distal section 38 could then be made of a super-elastic material that stretches readily in response to the same applied stress. The proximal and distal sections 36, 38 of the core-wire 26 could then have the same cross-sectional area but would nevertheless experience different strains when a tensile force is applied to the core-wire 26.

When a surgeon applies a proximally directed tensile force along the core-wire 26, that force causes a stress at each point on the core-wire 26. Because the distal section 38 of the core-wire 26 has a smaller cross-section than the proximal section 36 of the core-wire 26, the stress experienced by those points in the distal section 38 is greater than that experienced by those points in the proximal section 36. Since strain depends on stress, the distal section 38 of the core-wire 26 undergoes more strain than the proximal section 36 of the core-wire 26, and thus becomes significantly longer. This causes the distal section 38 of the core-wire 26 to extend. In this extended state, the core-wire 26 no longer constrains the flexible section 18 to follow a coiled path. The flexible section 18 is thus free to revert to its equilibrium compressed state in which it extends along the axis, as shown in FIG. 5. In this configuration, the flexible section 18 is said to have assumed its “insertion shape.”

When the surgeon removes the proximally-directed longitudinal force, hereafter referred to as the “tensile force,” on the core-wire 26, the distal section 38 of the core-wire 26 reverts to its relaxed state in which it defines a coiled path. Because the core-wire 26 is anchored to the flexible section 18, it constrains the flexible section 18 to follow the coiled path, as shown in FIG. 4. In this configuration, the flexible section 18 is said to have assumed its “ensnaring shape.”

The actuator 24 can be a handle with a trigger 27 as shown in FIG. 1. In this embodiment, the trigger is mechanically linked to the core-wire 26 so that pulling the trigger applies a tensile force along the core-wire 26. However, other types of actuators can be used so long as they too cause a tensile force along the core-wire 26. For example, the core-wire 26 can have a handle attached to its proximal end 32, in which case pulling on the handle directly applies a tensile force on the core-wire 26 without any intervening mechanical linkage.

In those embodiments in which the core wire 26 is a wire made of a single material, the diameter of the wire varies along its length. The ratio of the cross-sectional areas of the proximal and distal sections 36, 38 of the core-wire 26 will depend on the material properties of the core-wire 26. The ratio is selected such that a suitable differential strain can be achieved with only a modest exertion of force. The diameters of the two sections of the core-wire 26 are such that the applied tensile force will be insufficient for the core-wire 26 to lose the memory of its remembered shape. In general, this means that the tensile force must be such that the distal section 38 is elongated by less than 8% of its relaxed length, and preferably within 2% to 7% of its relaxed length.

There exist a variety of methods for manufacturing a core-wire 26 having two or more sections that differ in their yield forces. In one method, a continuous wire made of a shaped-memory metal is ground to a smaller diameter to form the distal section 38. The distal section 38 is then heat-set to the desired shape. To achieve actuation of the core-wire 26, there must be a sufficient difference in the yield force of the proximal section 36 and the yield force of the distal section 38. This is achieved by ensuring that the ratio of the diameter of the proximal section 36 to that of the distal section 38 is about 1.35 or greater. For a core-wire 26 having a non-circular cross-section, this is achieved by ensuring that the ratio of the cross-sectional area of the proximal section 36 to that of the distal section 38 is about 1.8 or greater.

The actual transition from one state to another can be viewed as a wave traveling along the core-wire 26. The direction in which this wave travels can be controlled by controlling the taper of the transition between the proximal section 36 and the distal section 38. In the case of a taper as shown in FIGS. 4 and 5, the wave travels from the proximal section 36 to the distal section 38 when the surgeon pulls on the core-wire 26. Conversely, when the surgeon releases the core-wire 26, the wave again travels from the proximal section 36 to the distal section 38.

A surgical instrument 10 as described above is also described in U.S. Pat. Nos. 6,500,185 and 6,652,536, the contents of which are herein incorporated by reference.

Various modifications to the surgical instrument 10 include the following:

### 1. Snare Modified to Release Medication

In one variation, the flexible section 18 is used to deliver medication. This can be achieved by coating the flexible section 18 with a polymer and incorporating a drug into that polymer. Suitable drugs that may be embedded in a polymer include heparin, nitrous oxide, thrombolytic medications, and anti-platelet medications, Plavix Aspirin tissue plasmagen activator (“TPA”) Urokinase Retavase Reopro, Integrilin, and Angiomax.

A variety of polymers can be used as a coating. Exemplary polymers include hydrophilic polymers, such as polyvinyl alcohol, polyacrylic acid, polyacrylic acid amide polyethylene oxide, polyethylene glycol, copolymers of polyethylene oxide (“PEO”) and polypropylene oxide (“PPO”) with urethane, such as those sold under the trade name TECOGEL or TECOPHILIC by Thermedics Detection Inc. of 207 Lowell Street, Wilmington, Mass., and HYDROMED® copolymeric urethanes sold by Carbomedics of 1300 East Anderson Lane, Austin, Tex., that swell when in contact with bodily fluids, including blood, and release drug by a dissolution of the drug into the blood. Other substances suitable for use as a coating include hydrogels such as alginates, collagen, gelatin, albumin or dextran derivatives.

Also suitable for use as a coating are hydrophobic polymers. These hydrophobic polymers release lipophilic drugs by dissolution into lipids or other drug dissolving components. Exemplary polymers include polybutylacrylate copolymers, polystyrene isobutylene copolymers, poly styrene isobutylene styrene copolymers and styrene butadiene copolymers.

2. Snare with Capture-Area Enhancement

The flexible section 18 can also be modified by the addition of a capture-area enhancer to provide greater surface area, or capture area, for capture of blood clots or other objects. Such capture-area enhancers are configured to deploy when the flexible section 18 assumes its ensnaring shape.

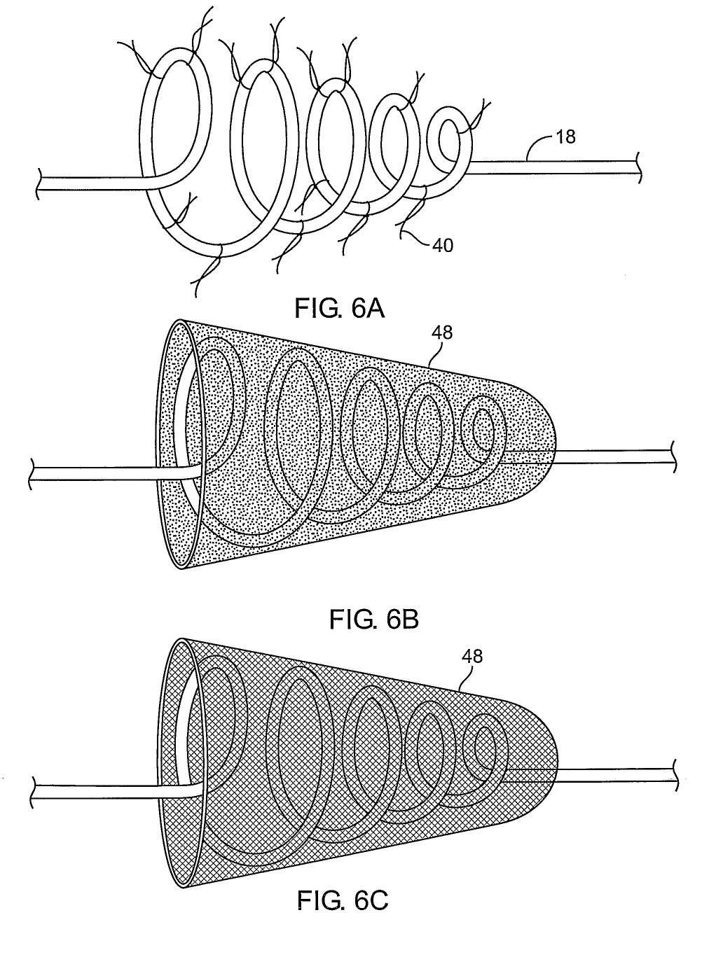

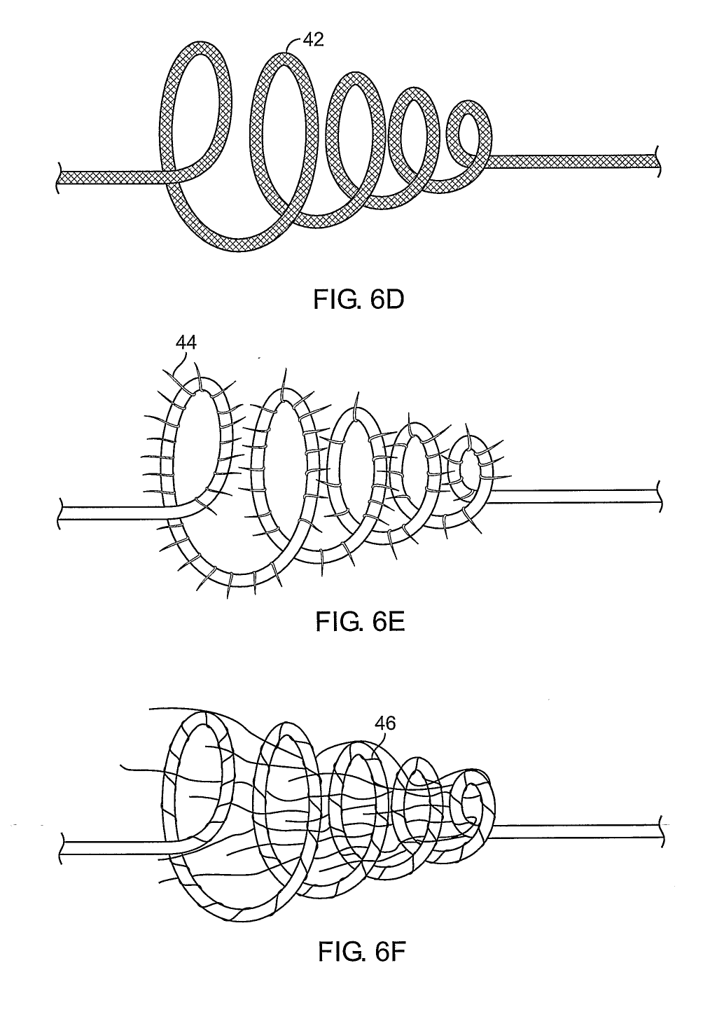

Examples of capture-area enhancers include threads 40, as shown in FIG. 6A, having one or both ends attached to the flexible section 18, and a coating 42 on the flexible section 18, made of a substance, such as a hydrogel that swells when placed in contact with water, as shown in FIG. 6D.

Other examples include a spiny coating 44, such as that shown in FIG. 6E. In addition to enhancing the capture area, the spines on the spiny coating 44 can spear a snared object, thereby enabling the flexible section 18 to more securely engage the snared object. In some embodiments, the spines are shaped specifically to hold the snared object more securely. Exemplary spines include barbed spines, or spines having forked tips that include two or more tines, which themselves can be barbed.

Other modifications include the placement of a sock 48 over the flexible section 18. Exemplary socks 48 include those made of nitinol mesh, as shown in FIG. 6C, and those made of a polymer, as shown in FIG. 6B. A suture 46, such as that used in closing wounds, can also be wound around the flexible section 18 to form a basket, or sock-like structure, as shown in FIG. 6F.

Capture-area enhancers such as those described above, spread the force exerted by a snared object on the flexible section 18 over a greater surface area. This reduces the likelihood that the snared object will disintegrate into smaller objects that elude the snare. Additionally, the capture-area enhancers shown herein effectively fill in gaps between adjacent turns of the flexible section 18, thereby increasing the likelihood that stray fragments that might otherwise escape through those gaps will ultimately be captured.

3. Snare with Multiple Core-Wires

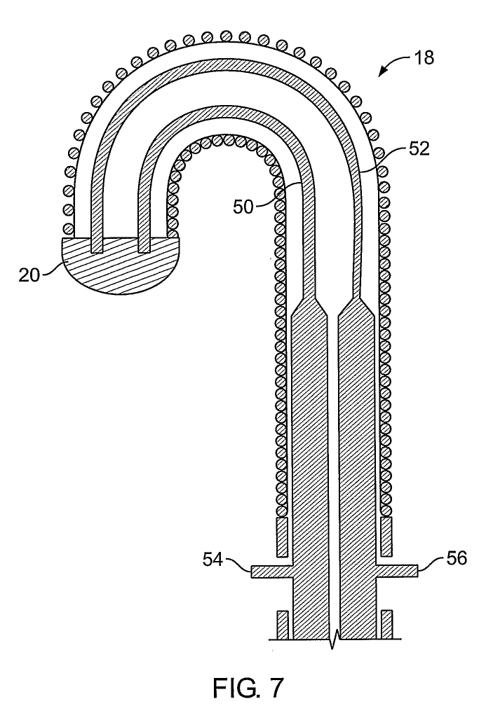

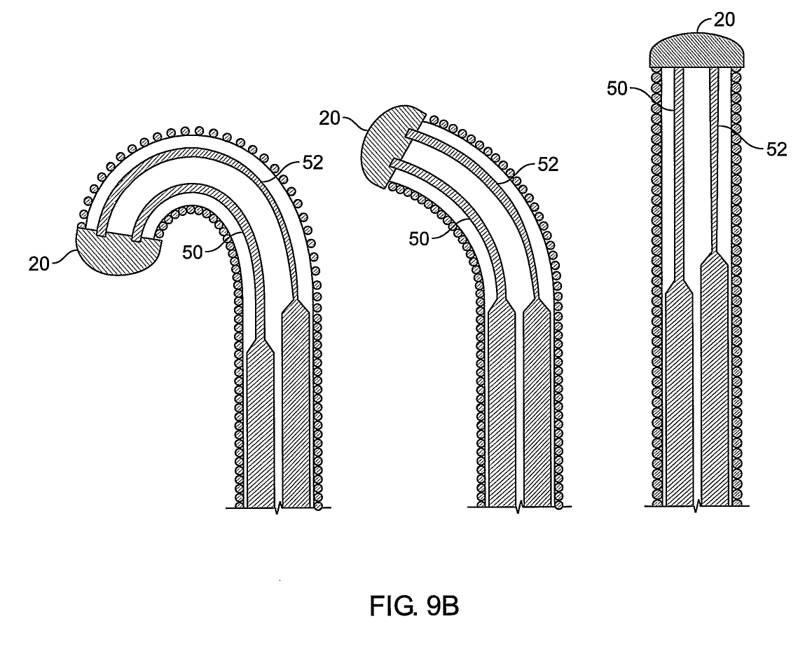

In another embodiment, shown in FIG. 7, the surgical instrument 10 includes first and second core-wires 50, 52, each of which is coupled to an associated actuator 54, 56. Both core wires 50, 52 are anchored to the end-cap 20. The two actuators 54, 56 are independently controlled, thereby allowing the two core-wires to transition between their two states independently of each other. As shown in FIGS. 9A-9C, the core wires 50, 52 can have different radii and can be positioned differently relative to each other. In addition, the core wires 50, 52 can have different tensile strengths, and/or different cross sections. Note that in FIG. 9A, σ refers to tensile strength, and A refers to cross-sectional area.

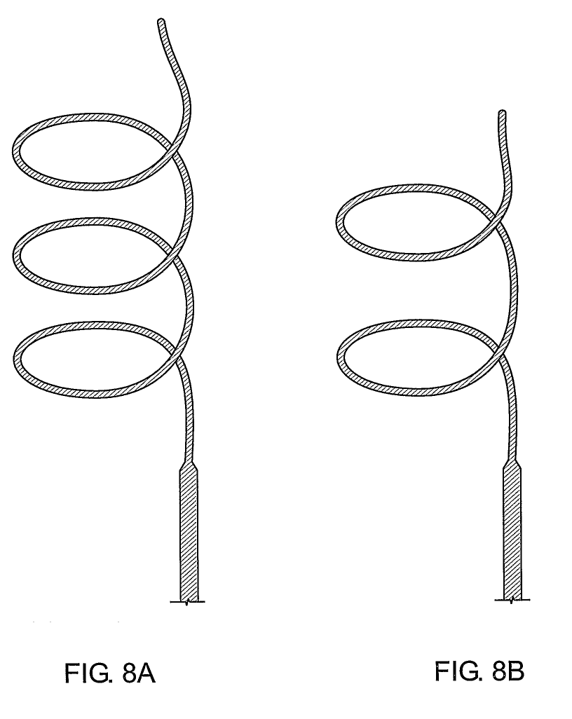

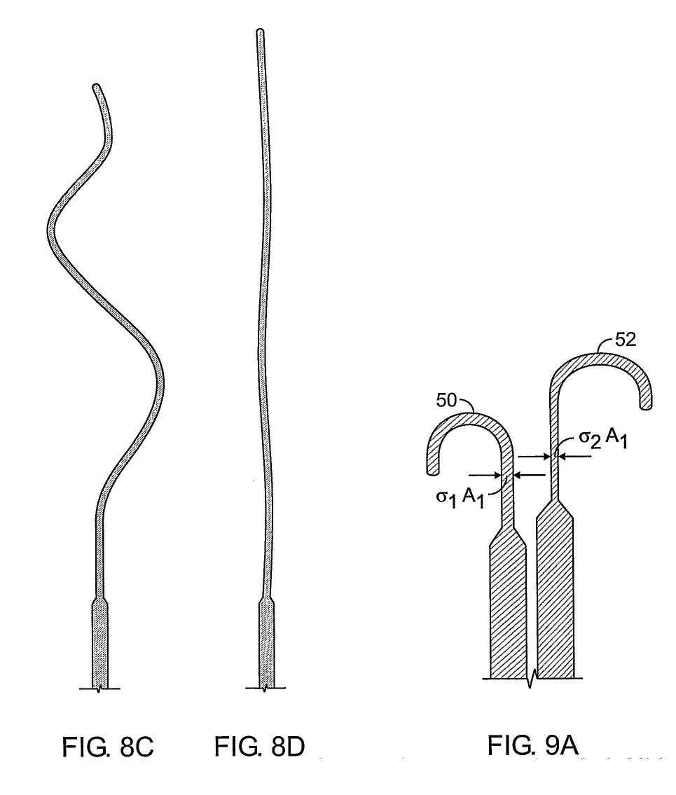

The configuration shown in FIG. 7 allows the flexible section 18 to define as many paths as there are combinations of core-wire states. In the illustrated example, two core-wires 50, 52 result in four such combinations. Consequently, the distal tip can assume four distinct paths, as shown in FIGS. 8A-8D.

The four different paths shown in FIGS. 8A-8D arise in response to different patterns of core wire actuation. In particular, FIG. 8A shows the tightly wound helix that results from relaxing both core wires 50, 52. FIGS. 8B and 8C are loosely wound helices resulting from relaxing one but not the other core wire. Since the two core wires 50, 52 have different stress/strain relationships, the shapes assumed by the flexible section 18 in FIGS. 8B and 8C differ depending on which of the two core wires 50, 52 is relaxed. In FIG. 8D, applying tension to both core wires 50, 52 straightens the flexible section 18.

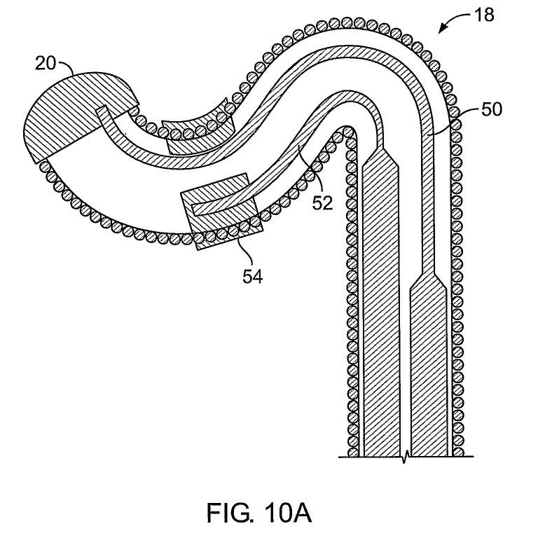

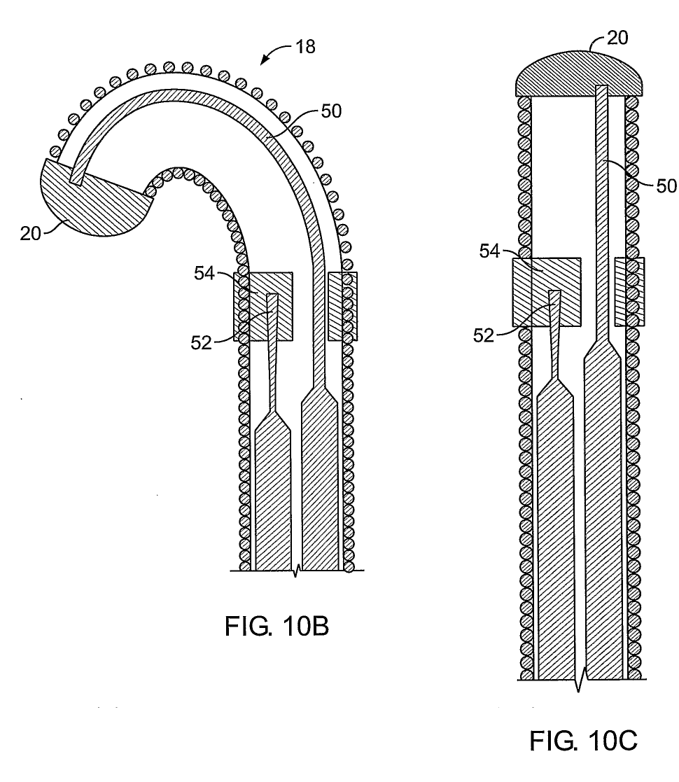

In some embodiments, the core wires 50, 52 have different lengths. For example, FIGS. 10A-10C show a first core-wire 50 extending all the way to the distal tip, where it is anchored to the cap 20, and a second core-wire 52 anchored to a collar 54 disposed part way to the end cap 20.

Multiple core wire embodiments, are useful for creating a grasping mechanism, such as a claw or an articulating member, that can assume several distinct shapes. Such embodiments provide increasingly prehensile snares.

FIG 10A shows the shape assumed by the flexible section 18 when both the first and second core-wires 50, 52 are relaxed. FIG. 10B shows the shape assumed by the flexible section 18 upon tensioning the second core-wire 52 while leaving the first core wire 50 relaxed. This causes the flexible section 18 to partially straighten itself. Finally, in FIG. 10C, both core-wires 50, 52 are under tension. This causes the flexible section 18 to completely straighten.

4. Snare with Loosely-Wound Distal Section

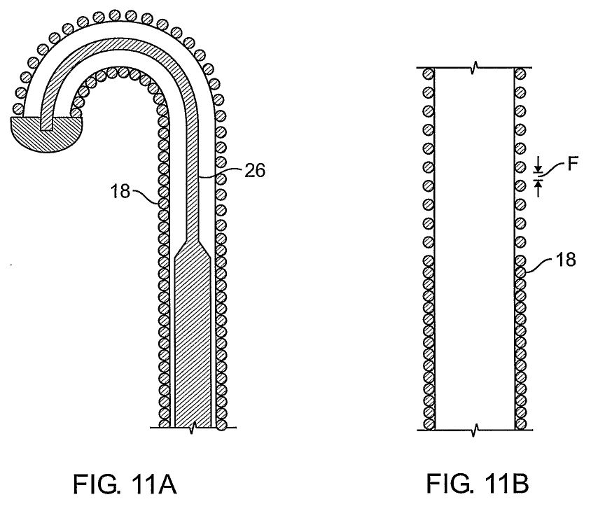

In some snares, the flexible section 18 tends to resist the core-wire 26 as the core-wire 26 transitions into its relaxed state. This difficulty arises when the flexible section is a close wound coil as shown in FIG. 11A.

To avoid this difficulty, certain embodiments of the snare feature a flexible section 18, as shown in FIG. 11B having a portion in which adjacent turns of the coil are separated by a gap F. The benefit of this configuration is that when in its extended state, the flexible section 18 aids the core-wire 26 in cycling to its relaxed position.

5. Guide Catheter with Expandable Distal Segment

To deliver a snare to a site at which a clot is located, one typically introduces the snare through a guide catheter. Typically, the guide catheter is barely wide enough to accommodate the snare with its flexible section 18 in the low-profile tensioned state.

Once the snare captures a clot, it is in its relaxed high-profile state. With this being the case, the flexible section 18 is too big to be retracted into the guide catheter. As a result, the guide catheter and the snare are removed with the flexible section 18 of the snare, and its captured clot, still hanging outside the catheter. During this removal procedure, there exists a risk that portions of the clot may break off and cause difficulties elsewhere in the patient.

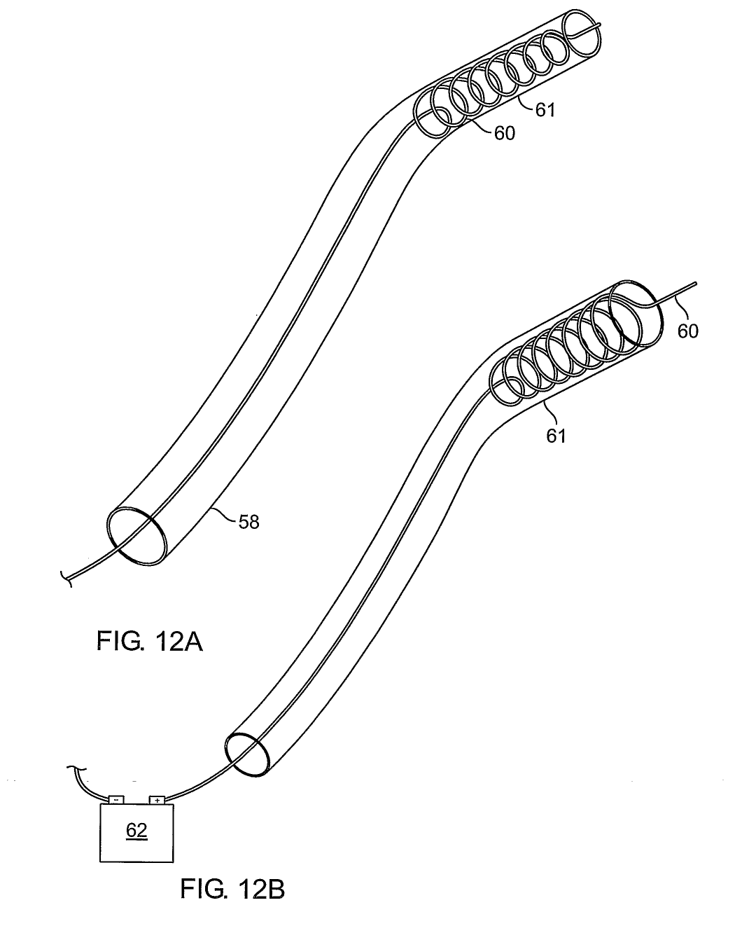

A guide catheter with an expandable distal segment circumvents this difficulty. One example of such a catheter, or other tubular member, as shown in FIGS. 12A-12B, includes a braided wire and polymer shaft 58 at the proximal region and a nitinol coil 60 encased in a low durometer elastomer 61 at its distal region. The nitinol coil 60 is a shape-memory alloy that, at room temperature, has an outer and inner diameter essentially equal to that of the braided shaft.

The nitinol coil 60 is connected to an electrical power source 63, such as a battery, that provides current for heating the coil 60. When the coil 60 is heated past its Aƒ temperature, it transitions into a super-elastic state. As the coil 60 is heated past its transition temperature, it assumes a new shape, show in FIG. 12B, that has been stored in its shape memory. This shape is one that has a diameter sufficient to accommodate the distal tip section of the snare in its relaxed state. The elastomer 61, in turn, expands as the coil 60 expands.

The elastomer 61 may be a drug eluting polymer with a time releasing capability for releasing any combination of the drugs described above.

6. Snare with Variable Strength Distal Segment

Another difficulty associated with withdrawal of an ensnared clot is that the core-wire 26 in its relaxed state has a tendency to stretch when subjected to relatively small forces. As a result, in the course of withdrawing the ensnared clot, it is possible for the snare to deform when it encounters an obstacle. This may result in loosening the grip that the snare has on the clot, which in turn may cause portions of the clot to escape.

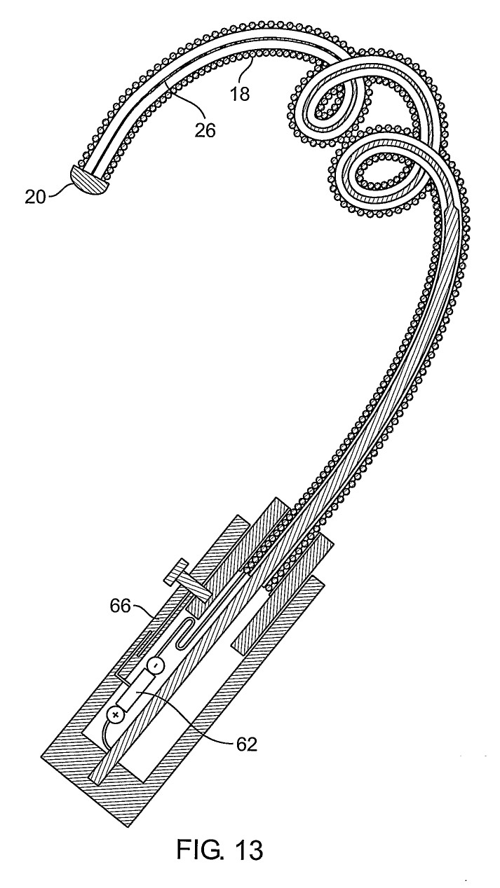

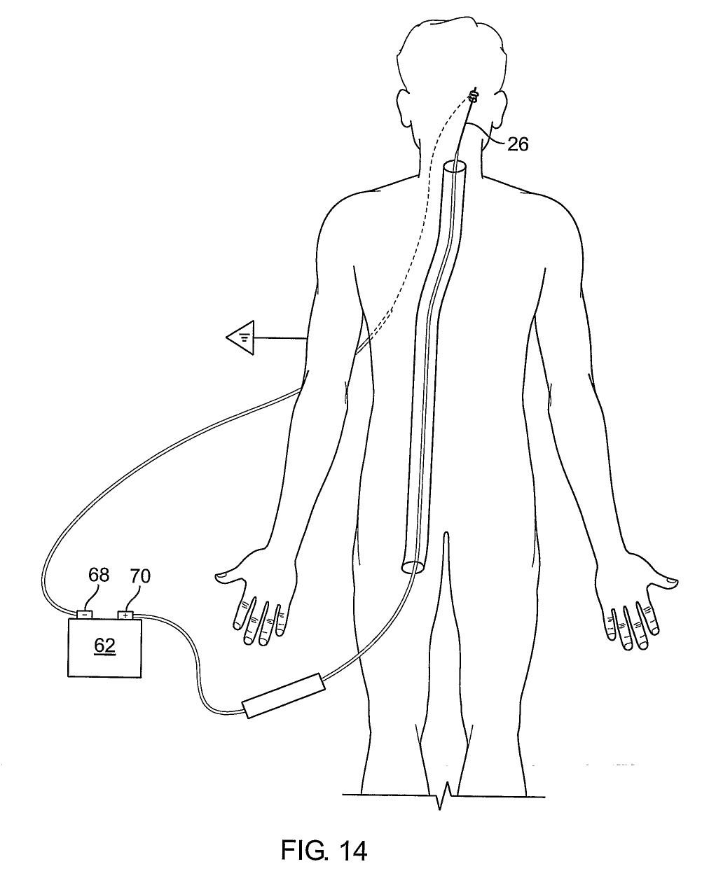

To avoid this difficulty, it is desirable to maintain the snare in its relaxed state, but to strengthen the material so that it does not readily deform when in its relaxed state. In the snares shown in FIGS. 13 and 14, this is achieved by passing a current through the core-wire 26. Since the shape memory alloy has a high resistivity, there is significant ohmic heating due to the current. This ohmic heating is greatest where the resistance of the core-wire 26 is greatest, i.e. at the tip where the core-wire is relatively narrow. The current thus heats the distal section 38 of the core-wire 26 to past its Aƒ temperature, thereby causing it to more effectively resist deformation.

To use the snare, one turns the current off and ensnares the clot. Then, with the clot safely ensnared, one allows the current to flow, thereby heating the distal section 38 of the core-wire 26 past its Aƒ temperature and causing the distal tip to stiffen. The snare, with the ensnared clot, is then withdrawn.

A suitable snare device has a nitinol corewire 26 with an Aƒ near 40-41 degrees C and a cross-sectional area that is smaller at the distal region than at the proximal region.

During insertion and delivery, the tip is made straight. In this configuration, it is desirable for the distal section to have low stiffness so as to be atraumatic.

A core-wire 26 can be made to have this property by reducing the product of the core-wire cross-sectional area and the tensile loading plateau. However, doing so compromises the snare's ability to grab and retrieve.

To negate this compromise, heat may be applied to the distal, coiled segment of the nitinol core-wire 26, resulting in a coiled core-wire 26.

One way to heat the core-wire 26 is to provide a switch 68 to connect a battery 62 between the support member, e.g. the flexible section 18, and the core-wire 26, as shown in FIG. 13. The portion of the core-wire 26 having greatest resistance, i.e. the smallest diameter portion of the core-wire 26 would heat the most. In this configuration, the support member and the core-wire 26 would need to be electrically isolated from each other except at the point of connection, which would be coincident with the point of mechanical anchoring.

Another way to heat the core-wire 26 is to use the patient as part of the circuit, as shown in FIG. 14. In this case, a first terminal 68 of the battery is connected to the patient and a second terminal 70 is connected to the core wire 26. This configuration eliminates the need to electrically isolate the support member from the core-wire 26.

Having described the invention, and preferred embodiments thereof, what we claim is:

1. A snare comprising:
a support defining an axis, the support including a flexible section;
a capture-area enhancer coupled to the flexible section;
a core-wire extending along the axis and anchored to the flexible section, the core-wire having a first state, in which it defines a first path and a second state, in which it defines a second path; and
an actuator engaged to a proximal end of the core-wire for selectively causing the core-wire to transition between the first state and the second state.

2. The snare of claim 1, wherein the first state of the core-wire is a relaxed state and the second state of the core-wire is a tensioned state.

3. The snare of claim 1, wherein the capture-area enhancer comprises a plurality of threads attached to the flexible section.

4. The snare of claim 1, wherein the capture-area enhancer comprises a sock covering the flexible section.

5. The snare of claim 4, wherein the sock comprises a polymer.

6. The snare of claim 4, wherein the sock comprises a nitinol mesh.

7. The snare of claim 1, wherein the capture-area enhancer comprises a hydrogel coating on the flexible section.

8. The snare of claim 1, wherein the capture-area enhancer comprises a suture wound on the flexible section.

9. The snare of claim 1, wherein the capture-area enhancer comprises a spiny coating on the flexible section.

10. The snare of claim 1, further comprising a drug-releasing polymer coating on the flexible section.

11. The snare of claim 1, further comprising:
an additional core-wire extending along the axis and anchored to the flexible section, the additional core-wire having a first state, in which it defines an additional first path, and a second state, in which it defines an additional second path; and
an additional actuator engaged to a proximal end of the additional core-wire for selectively causing the additional core-wire to transition from the first state to the second state.

12. The snare of claim 1, further comprising a power source in electrical communication with the core wire, the power source providing current to heat the core wire, thereby causing the core wire to transition between the first and second state.

13. A snare comprising:
a support defining an axis, the support having a flexible distal section having an uncompressed state and a compressed state, the flexible distal section defining a first path relative to the axis when in the compressed state;
a capture-area enhancer coupled to the distal section;
a core-wire extending along the axis and anchored to the flexible distal section, the core-wire having a relaxed state and a tensioned state, the core-wire defining a second path relative to the axis when in the relaxed state; and
an actuator engaged to a proximal end of the core-wire for selectively applying a tensile force thereto, the tensile force causing the core-wire to transition from its relaxed state, in which the flexible distal section is in its uncompressed state, to its tensioned state, in which the flexible distal section is in its compressed state.

14. The snare of claim 13, wherein the capture-area enhancer comprises a plurality of threads attached to the distal section.

15. The snare of claim 13, wherein the capture-area enhancer comprises a sock covering the distal section.

16. The snare of claim 13, wherein the capture-area enhancer comprises a hydrogel coating on the distal section.

17. The snare of claim 13, wherein the capture-area enhancer comprises a suture wound on the distal section.

18. The snare of claim 13, wherein the capture-area enhancer comprises a spiny coating on the distal section.

19. The snare of claim 13, further comprising a drug-releasing polymer coating on the distal section.

20. A surgical snare for ensnaring an object in a patient, the snare comprising:
a flexible section that selectively switches between an insertion shape and an ensnaring shape; and
a capture-area enhancer configured to deploy when the flexible section assumes the ensnaring shape.

21. The surgical snare of claim 20, wherein the capture-area enhancer comprises a plurality of threads coupled to the flexible section.

22. The surgical snare of claim 20, wherein the capture-area enhancer comprises a sock covering the flexible section.

23. The surgical snare of claim 20, wherein the capture-area enhancer comprises a hydrogel coating of the flexible section.

24. The surgical snare of claim 20, wherein the capture-area enhancer comprises a suture wound on the flexible section.

25. The surgical snare of claim 20, wherein the capture-area enhancer comprises a spiny coating of the flexible section.
